(19) United States
(12) Patent Application Publication
(10) Pub. No.: US 2012/0283686 A1
(43) Pub. Date: Nov. 8, 2012

(54) Diaper With Fecal Matter Collector

(76) Inventor: Shawn Ramage, Lucerne Valley, CA (US)

(21) Appl. No.: 13/462,742

(22) Filed: May 2, 2012

Related U.S. Application Data
(60) Provisional application No. 61/481,913, filed on May 3, 2011.

(51) Int. Cl. A61F 13/515

(52) U.S. Cl. 604/385.19

(57) ABSTRACT

A disposable diaper for use on babies having an outer barrier layer which is impervious to liquids, an inner barrier layer which comes in contact with a baby's skin, and an intermediate absorptive layer is provided. In one embodiment, the new diaper includes longitudinal skirts for preventing feces from migrating outward of the diaper. In another embodiment, the new diaper is configured into an upper region and a lower region. The upper region captures a baby's urine discharges while the lower region includes a pouch for the capture of feces. The upper and lower regions are separated to prevent the communication of liquid and solid matter between the regions.

### CROSS-REFERENCE TO RELATED APPLICATIONS

This patent application claims the benefit of U.S. Provisional Patent Application Ser. No. 61/481,913 filed May 3, 2011 for a Diaper With Fecal Matter Collector, which application is incorporated in its entirety herein by this reference.

### FIELD OF INVENTION

This invention relates to a disposable diaper. More particularly, it relates to a disposable diaper having separate portions for the receipt of fecal matter and for the absorption of urine.

### BACKGROUND OF THE INVENTION

In the past a wide variety of disposable baby diapers have been presented and such disposable diapers have gained widespread acceptance in the commercial market. Such diapers have conventionally included a facing layer to be brought into contact with an infant's skin, an absorbent panel adjacent thereto, and a water-impervious or a water repellant outer layer. Known types of disposable diapers have many functional deficiencies. Some lack absorptive capacity and, most, if not all, lack any provision for keeping feces away from contact with an infant's skin. Prolonged contact of feces with an infant's or user's skin may have adverse health consequences such as rashes or infections. What is needed therefore is an improved diaper that provides for sufficient absorptive capacity for a baby's urine discharges and has substantial ability to keep feces separated from a baby's skin.

### SUMMARY OF THE INVENTION

Accordingly, the present invention is directed towards providing a disposable diaper which can effectively solve the aforementioned problems of the prior art. Briefly stated, the present invention provides a diaper with an outer barrier layer which is impervious to liquids, an inner barrier layer which comes in contact with a baby's or user's skin which may also be impervious to liquids and, disposed between the inner and outer layers is an intermediate layer of absorptive material. In one embodiment of the new diaper, inner and outer longitudinal skirts are attached to the inner layer of the diaper. The skirts function to keep feces within the diaper and therefore don't allow the feces to migrate to the baby's legs or other body parts. In this manner, the skirts greatly ease cleaning a baby when changing a diaper.

In another embodiment of the present invention, the diaper is split into an upper region and a lower region which-are separated by a barrier or separator. The upper region captures a baby's urine discharges while the lower region includes a pouch for the capture of feces. The separator prevents communication of liquid or solid matter between the two regions. In the upper region, the semi-permeable inner barrier layer allows for urine to pass through to the absorptive material of the intermediate layer. In the lower region, the intermediate layer is formed with a pouch for the containment of feces and an opening is provided in the inner barrier layer to allow for the feces to pass into into the pouch.

These and other features of the new invention will become more apparent from the following detailed description taken in conjunction with the accompanying drawings.

### BRIEF DESCRIPTION OF DRAWINGS

FIG. 1 is a top view of one embodiment of the diaper of the present invention, i.e., a view showing the interior of the diaper that is in contact with the baby's skin when the diaper is wrapped around a baby.

FIG. 2 is a top view of another embodiment of the diaper of the present invention, i.e., a view showing the interior of the diaper that is in contact with the baby's skin when the diaper is wrapped around a baby.

### DETAILED DESCRIPTION OF THE PREFERRED EMBODIMENTS

For a more complete understanding of the present invention, reference is now made to the following description of various illustrative and non-limiting embodiments thereof, taken in conjunction with the accompanying drawings in which reference numbers indicate some of the features of the invention.

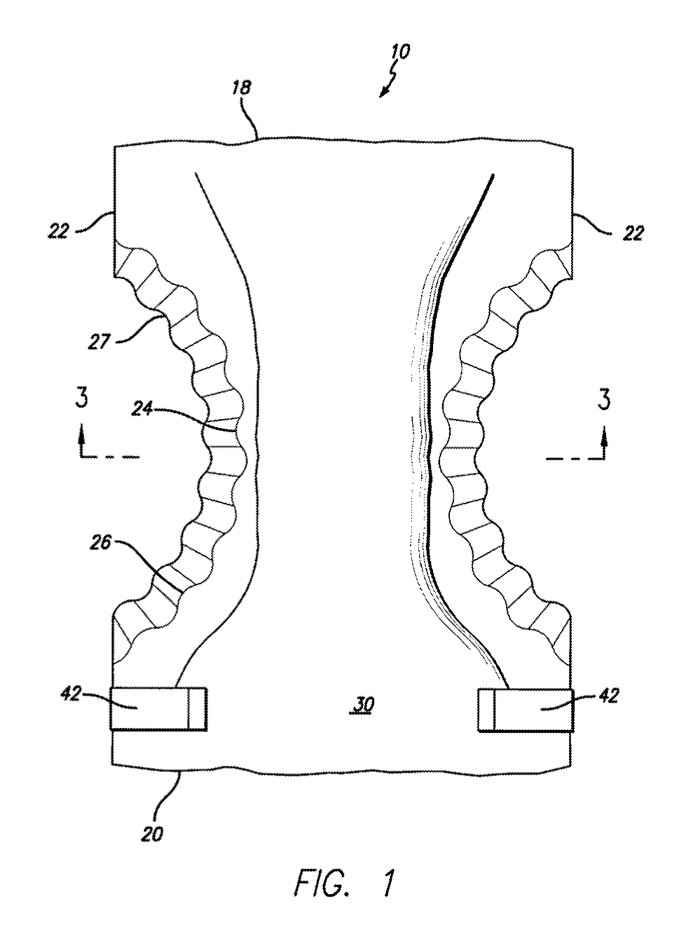

Referring to FIG. 1, in one embodiment of the present invention disposable diaper, the disposable diaper 10 has a generally rectangular shape defined by an upper edge 18 and a lower edge 20 and right and left side edges 22. The diaper has a center portion 24 which has generally inwardly curving side edges 26. The inwardly curving edges 26 are designed to fit around a baby's legs and thereby provide a snug fit of the diaper to the baby. The diaper is comprised of three material layers, an outer barrier layer 28 (not shown), an inner barrier layer 30 and an intermediate absorbent layer 32. Spaced about the diaper's longitudinal axis and parallel to that axis are right and left longitudinal skirts 27 and 29. The longitudinal skirts are attached to the inner layer 32 and extend upwardly from the inner layer 32. The skirts seal a region 31 around the baby's anus and thereby prevent feces from migrating outside of the diaper. In so doing, the skirts make cleaning the baby when changing a diaper a substantially easier task because the baby's feces are constrained within the longitudinal area 31 between the skirts.

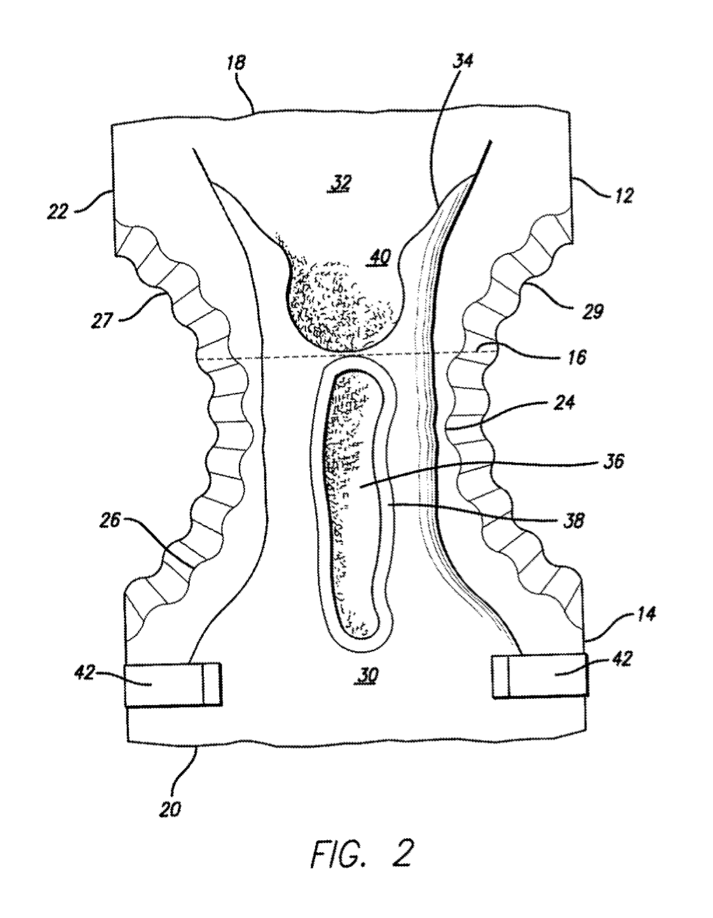

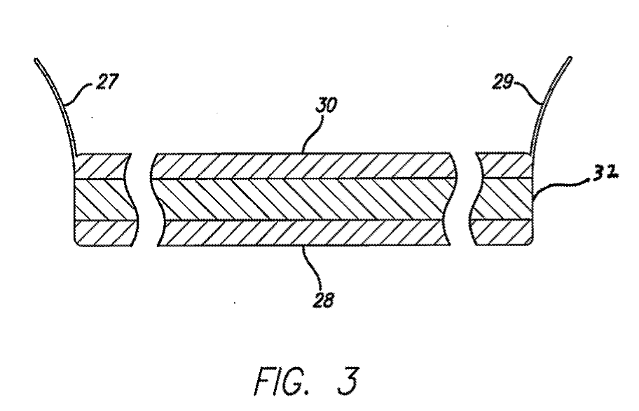

Referring now to FIG. 2, in another embodiment of the present invention, the disposable diaper 10 also has a generally rectangular shape defined by an upper edge 18 and a lower edge 20 and right and left side edges 22. The diaper likewise has a center portion 24 which has generally inwardly curving side edges 26. The inwardly curving edges 26 are designed to fit around a baby's legs and thereby provide a snug fit of the diaper to the baby. The diaper is also comprised of three material layers, an outer harrier layer 28 (not shown), an inner barrier layer 30 and an intermediate absorbent layer 32.

In the embodiment disclosed in FIG. 2, the present invention diaper further includes an upper region 12 and a lower region 14. The upper and lower regions are separated by a separator 16. The upper region 32 is semi-permeable and allows for urine to pass through to the absorbent intermediate layer 32. In an alternative construction, the inner barrier layer 33 may be cut away to expose the absorbent intermediate layer 32. The cut-away portion of the alternative embodiment is defined by edge 34 which defines a urine absorption region 40.

With continued reference to FIG. 2, the lower region 14 includes a cavity or pouch 36 formed in the intermediate layer 32. The cavity 36 is accessible from the inner layer 30 of the diaper 10 via an opening 38 in the inside barrier layer 30. The cavity 36 is generally centered laterally in lower region 14 of the diaper 10 and is generally rectangular in shape. The cavity 36 functions as a collection pouch for a baby's feces and therein helps to keep the feces from prolonged contact with the baby's skin. Prolonged contact of feces with a baby's skin can cause rashes, infections and may otherwise adversely affect the health of the baby.

The barrier or separator 16 prevents communication of liquid or solid matter between the upper region 12 and the lower region 14. Depending upon whether natural or synthetic materials are used for the inner 30, outer 28 and intermediate 32 layers, the separator may be as simple as a line of stitching between the upper 12 and lower 14 regions or in the case of plastic materials could be formed by heat sealing the layers. The separator 16 could also be a panel between the inner and outer layers 12 and 14.

The diaper is attachable around the waist of the baby by means of fastening tabs 42 located along the upper and lower edges 18 and 20 of the diaper. The fastening tabs 42 may be adhesive tabs, Velcro tabs or the like. Variations of the diaper may be used by adults who suffer from incontinence in which case belt loops (not shown) may be substituted for the fastening tabs 42.

The outer barrier layer 28 is generally either impervious or semi-permeable to liquids and will typically be made of a plastic material. The inner barrier layer 30 is also generally semi-permeable to liquids, but in alternative embodiments may be impervious to liquids. The absorbent intermediate layer 32 may be formed from a variety of absorptive materials including cotton batting, plastic batting and other like materials. Suitable materials for the inner 30 and outer 28 barrier layers and the intermediate layer 32 are well known in the art. Methods of attaching the layers to together such as adhesive bonding, heat sealing or stitching are also well known in the art.

In the upper region 12, the inner harrier layer 30 may he made of semi-permeable material to allow for urine to pass through to the absorbent layer 32, or alternatively, the inner barrier layer maybe cut away to expose the absorptive material of the intermediate layer 32. In the lower region, a pouch 36 is formed in the intermediate layer 32 for the containment of feces and inner barrier layer 30 formed to include an opening 38 into the pouch. The inner barrier layer 30 will typically be fused to the intermediate layer 32 which prevents the opening 38 in the intermediate layer from being displaced from the cavity 36.

The foregoing detailed description and appended drawings are intended as a description of the presently preferred embodiment of the invention and are not intended to represent the only forms in which the present invention may be constructed and/or utilized. Those skilled in the art will understand that modifications and alternative embodiments of the present invention which do not depart from the spirit and scope of the foregoing specification and drawings, and of the claims appended below are possible and practical. It is intended that the claims cover all such modifications and alternative embodiments.

1. A disposable diaper comprising:
inner barrier layer, an outer barrier layer, and an intermediate layer of absorbent material disposed therebetween;
the diaper being generally rectangular having short sides and long sides, the long sides have an inwardly curving center portion to provide for a snug fit around a user's legs;
the diaper being separated into an upper region and a lower region;
the upper region having absorbent material for the collection of urine, wherein the inner barrier layer of the upper region is formed from a semi-permeable material to allow for urine to pass through the inner barrier layer to the absorbent intermediate layer;
the lower region including a pouch for the collection of feces, wherein the pouch for the collection of feces comprises a cavity formed in the absorbent intermediate layer, the cavity being accessible via an opening formed in the inner barrier layer.

2. The disposable diaper of claim 1, wherein the upper and lower regions are separated by a separator which prevents the communication of liquid or solid matter between the two regions.

3. The disposable diaper of claim 1, wherein the outer barrier layer is made of a material impervious to liquids.

4. A disposable diaper comprising:
an inner and outer barrier layers and an intermediate layer of absorbent material therebetween;
inner and outer longitudinal skirts attached to the diaper which prevent feces from migrating outwardly of the diaper.

5. The disposable diaper to claim 4, wherein the diaper includes a center portion, the center portion including inwardly curving side edges, wherein the inwardly curving side edges provide a snug fit of the diaper around a baby's legs and thereby prevent the migration of feces outside of the diaper.

6. The disposable diaper of claim 4, wherein the upper and lower regions are separated by a separator which prevents the communication of liquid or solid matter between the two regions.

7. The disposable diaper of claim 4, wherein the inner barrier layer is made of a material semi-permeable to liquids.

8. The disposable diaper of claim 4, wherein the inner barrier layer is partially cut away to reveal the absorbent intermediate layer of the diaper.

9. A disposable diaper comprising:
inner and outer barrier layers and an intermediate layer of absorbent material therebetween;
the diaper being separated into an upper region and a lower region;
the upper region having absorbent material for the collection of urine; and the lower region including a pouch for the collection of feces.

10. The disposable diaper of claim 9, wherein the pouch for the collection of feces comprises a cavity formed in the absorbent intermediate layer, the cavity being accessible via an opening formed in the inner barrier layer.

11. The disposable diaper of claim 9, wherein the inner barrier layer is made of a semi-permeable material to allow for urine to pass through to the absorbent material of the intermediate layer.

12. The disposable diaper of claim 9, wherein the inner barrier layer is partially cutaway in the upper region to expose the absorbent material of the intermediate layer for the absorption of urine.

13. The disposable diaper of claim 9, wherein the upper and lower regions are separated by a separator which prevents the communication of liquid or solid matter between the two regions.

14. The disposable diaper of claim 9, wherein the diaper is generally rectangular in shape and wherein the long edges have an inwardly curving center portion to provide for a snug fit around a user's legs.

15. The disposable diaper of claim 9, wherein the outer barrier layer is made of a material impervious to liquids.

16. The disposable diaper of claim 9, wherein the inner barrier layer is made of a material semi-permeable to liquids.

17. The disposable diaper of clam 9, wherein the diaper further includes adhesive tabs for attaching the diaper around a user's waist.

18. The disposable diaper of clam 9, wherein the diaper further includes Velcro tabs for attaching the diaper around a user's waist.
